(19) United States
(12) Patent Application Publication
(10) Pub. No.: US 2011/0009930 A1
(43) Pub. Date: Jan. 13, 2011

(54) INFLATABLE THERMAL BLANKET

(75) Inventors: Arthur Everardus OFFICIER, Epe (NL); Berend Jan TEUNISSEN, Haaksbergen (NL)

Correspondence Address: KNOX PATENTS, P.O. BOX 30034, KNOXVILLE, TN, 37930-0034, US

(73) Assignee: THE SURGICAL COMPANY HOLDING B.V., Amersfoort (NL)

(21) Appl. No.: 12/501,661

(22) Filed: Jul. 13, 2009

(51) Int. Cl. A61F 7/00

(52) U.S. Cl. 607/104

(57) ABSTRACT

An inflatable thermal blanket for providing conditioned air to a human or animal body positioned on top of thermal blanket,
a base sheet of an air impermeable material, in use remote from the body;
a top sheet of an air permeable material, in use in contact with the body;
a peripheral connection joining the base sheet and top sheet to define an air chamber;
multiple connections distributed within the peripheral connection across interior surface regions of the base sheet and top sheet and connecting the base sheet and top sheet, and
an inlet port for providing the conditioned gas to the inflatable chamber.

### BACKGROUND

The present invention relates to a blanket used to covering at least a portion of the body of a human, or other animal, in order to bath the body portion in a conditioned gas. More specifically, the present invention is related to an inflatable thermal blanket for providing a conditioned air to the body of a user.

Inflatable thermal blankets are commonly used as so called upper body blankets. They are positioned on top of or over a body or portion of a body. These blankets are often used in hypothermia situations, pre-, peri- or post-operative. In these thermal blankets, warm air is inserted from a hyperthermia unit, though a supply hose, into the inlet port of the blanket. After inflating the blanket, this warm air is communicated though a distribution means in the blanket to a body or portion of a body. Usually, an air permeable layer is used for releasing the warm air to the body.

These known blankets are placed over a person in order to keep them warm. During surgery or other medical treatments when access to various parts of the body is needed, there is need for a device for keeping a body conditioned.

### SUMMARY OF THE INVENTION

The invention aims to provide a new use to known thermal blankets.

In particular, the inventions seeks to provide a device and method for conditioning a body while allowing access to the body.

According to a first aspect of the invention this is realized with an inflatable thermal blanket for providing conditioned air to a human or animal body positioned on top of said thermal blanket, said thermal blanket comprising:
a base sheet of an air impermeable material, in use remote from said body;
a top sheet of an air permeable material, in use in contact with said body;
a peripheral connection joining said base sheet and top sheet to define an air chamber;
multiple connections distributed within said peripheral connection across interior surface regions of said base sheet and top sheet and connecting said base sheet and top sheet, and
an inlet port for providing the conditioned gas to said inflatable chamber.

The invention furthermore provides a method for conditioning a person, in particular the temperature of a patient, comprising the steps of:
providing a blanket on a surface, said blanket comprising:
   a base sheet of an air impermeable material, in use remote from said body;
   a top sheet of an air permeable material, in use in contact with said body;
   a peripheral connection joining said base sheet and top sheet to define an air chamber;
   multiple connections distributed within said peripheral connection across interior surface regions of said base sheet and top sheet and connecting said base sheet and top sheet, and
an inlet port for providing the conditioned gas to said inflatable chamber, and
positioning the person on said blanket.

The present invention provides an inflatable thermal blanket capable of providing a gas, such as for example heated air, to for instance the underbody of a user. The thermal blanket includes an inflatable portion for receiving the conditioned gas under pressure and for being positioned under the body of a user. In other words, a body is positioned on top of the thermal blanket. The inflatable portion is defined by an air impermeable base sheet and an air permeable top sheet which is being directed to the body. In an embodiment, it may comprise one or more air permeable barrier sheets positioned between the base sheet and top sheet. The inflatable portion also includes an inlet port, being connected to a source of conditioned gas.

This thermal blanket and method of the invention provides the possibility of keeping a person or animal warm, for instance during medical treatment. The blanket of the invention is for instance used during surgery. Clinical studies showed that using the blanket of the invention provides the most effective way of keeping a body warm. In fact, it has the highest efficiency in comparison to other methods. Additionally, the blanket of the invention is disposable, in contrast to other means for warming a body or keeping a body warm. This makes the use in medical environment in particular easy.

In an embodiment, the top sheet has an air permeability of 1-400 mm/sec measured at a pressure drop of 100 Pa. This, the diffusion of air is further optimized in this type of blanket.

In an embodiment, the inlet port is positioned asymmetrically compared to the longitudinal axis of the blanket.

In an embodiment, the inlet port is connected to a dual inlet channel, to in use split the flow to either side of the blanket.

In an embodiment, the splitted flow is directed by an non-inflatable shaped area, positioned where in use the head is located. In an embodiment, it is triangularly shaped. In a further embodiment, it has rounded edges or corners. Thus, in use the flow of air is split in two mayor directions.

In an embodiment, the splitted flow is dispersed to an outer channel and a central part, covering the body. In an embodiment, the outer channels and the central part comprise open contacts at both the upstream and downstream side of the outer channels. Thus, conditioned gas can be diffused and is allowed to exit over the entire area of the blanket.

In an embodiment, the central part comprises multiple seal connections between the base sheet, barrier sheet(s) and top sheet such as dots, strips, channels etc. Thus, the layers of the blanket are connected to one another.

In an embodiment, the total blanket length varies at fixed or stretched slit lengths, whilst multiplying the number of multiple connections, related to the blanket length increase.

In an embodiment, the base and/or top sheets extend outward beyond said peripheral connection.

In an embodiment, the inflatable blanket further has one or more barrier sheets of an air permeable material sandwiched between said base sheet and said top sheet. In an embodiment, the barrier sheet or barrier sheets extend over substantially the entire area enclosed by the peripheral connection. The barrier sheet improves the diffusion of conditioned gas through the area of the blanket. In an embodiment, the one or more barrier sheets is or are attached to said top sheet and said base sheet. In an embodiment, the barrier sheet has an air permeability of 1-400 mm/sec measured at a pressure drop of 100 Pa. This permeability provides an improved diffusion even when a body is placed on top of the blanket.

In an embodiment, the inflatable thermal blanket further comprises openings through said top sheet and base sheet to transfer arms through said thermal blanket. In an embodiment, the openings comprise slits. In an embodiment, the openings comprise slits which are selectively openable. In an embodiment, the openings comprise slits which are selectively openable through a perforated tear strip, taping, folding, sealing, snapping, or a combination thereof. In an embodiment, the slits are initially sealed closed and are reversibly openable. In an embodiment, the slits are permanently open. In an embodiment, the slits have rounded ends. Provision of the openings allow one or two arms to be extended through the blanket. Thus, one or two arms become available, for instance for infusion or monitoring for instance blood pressure. Furthermore, it facilitates wrapping of the blanket around a body. In an embodiment, the slits have a length of about 30-80 cm. In an embodiment, the various sheets of the blanket can be attached to one another at two longitudinal strips, substantially parallel to part of the peripheral connection. Within the area of the longitudinal strip, a provision can be provided for making a through opening in the blanket. This can be a permanent opening, for instance via a tear strip or a perforation line. The opening can also be opened and closed. To that end, a tape or adhesive strip or for instance Velcro strip can be provided.

In an embodiment, the inflatable thermal blanket further comprises fixing provisions on the longitudinal sides of the inflatable thermal blanket, allowing said longitudinal end to be held towards one another. In an embodiment, the fixing provisions are adapted to adhere to the body, in particular said fixing provisions include tape. The fixing provisions allow wrapping or at least partial wrapping of the blanket around a body. Clinical tests showed that this improved conditioning.

In an embodiment, the base sheet comprises a heat reflective layer. To that end, for instance an aluminium coating layer can be provided, or another heat reflective layer which is known from other fields of art.

In an embodiment of the method, it further comprising the step of folding longitudinal ends of said blanket toward one another after said person is positioned on said blanket.

In an embodiment of the method, the blanket comprises openings near said longitudinal end, and said method further comprises the step of taking the arms of the person through the openings before folding said longitudinal ends.

In an embodiment of the method, the blanket further comprises fixing provisions for holding said longitudinal ends near one another after folding them towards one another, said method further comprising the step of fixing said fixing provisions for fixing said longitudinal ends of said blanket positioned near one another.

The various aspects discussed in this patent can be combined in order to provide additional advantages. Furthermore, separate aspects disclosed in this description and/or in the claims may form the basis of one or more divisional patents.

### DESCRIPTION OF THE DRAWINGS

The invention will be further elucidated referring to an embodiment of a thermal blanket and its use shown in the attached drawings, showing in:

FIG. 1 a top view of a small version of an inflatable thermal blanket of the present invention;

FIG. 2 a top view of a tall version of an inflatable thermal blanket of the present invention;

FIG. 3 a projection of a human body at the small inflatable thermal blanket of the present invention, projecting the arms through the slits;

FIG. 4 a projection of a human body at the tall inflatable thermal blanket of the present invention, projecting the arms through the slits;

FIG. 5 the wrapping feature of the small version of an inflatable thermal blanket;

FIG. 6 the wrapping feature of the tall version of an inflatable thermal blanket;

FIG. 7 a cross section of a part of a blanket, and

FIG. 8 a cross section of another embodiment.

### DETAILED DESCRIPTION OF EMBODIMENTS

In the drawings, similar features have the same reference numbers.

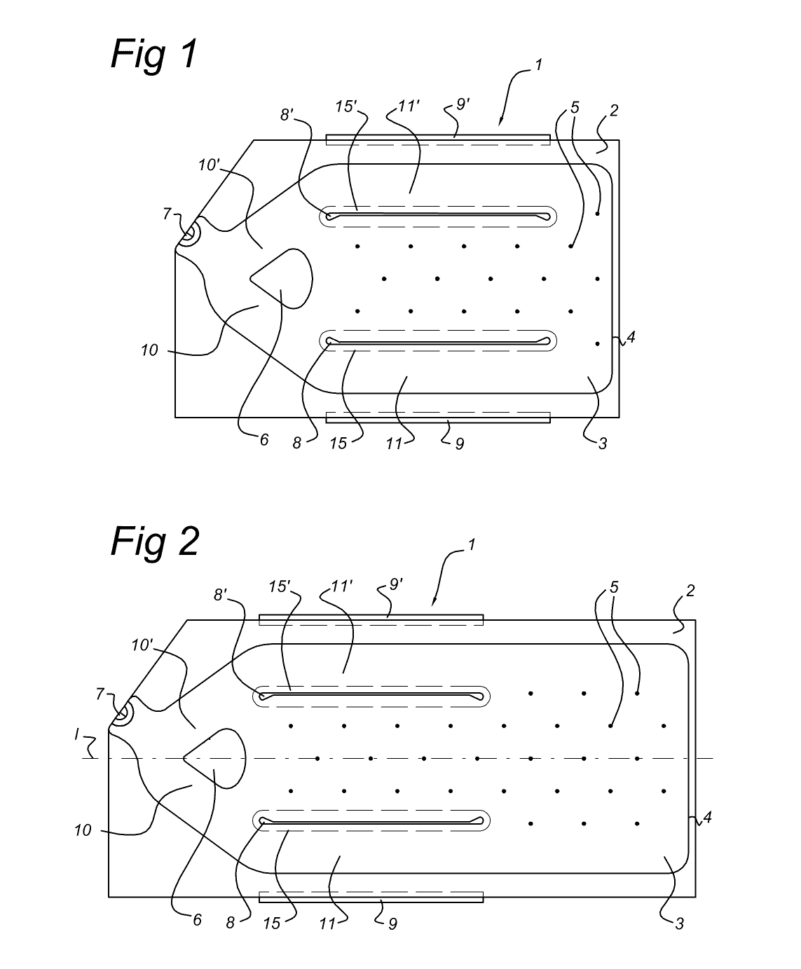

An embodiment of the thermal blanket of the present invention is illustrated generally in FIGS. 1 and 2, showing respectively a small and tall edition of the blanket 1.

Thermal blanket 1 of FIG. 1 has a base sheet 2. This base sheet 2 is of an air impermeable material. Attached to the base sheet 2 is a top sheet 3. These sheets 2, 3 are attached to one another via a peripheral connection 4. Thus, base sheet 2 and top sheet 3 attached to one another via peripheral connection 4 form an inflatable air chamber or inflatable portion. The inflatable portion has an inlet port 7.

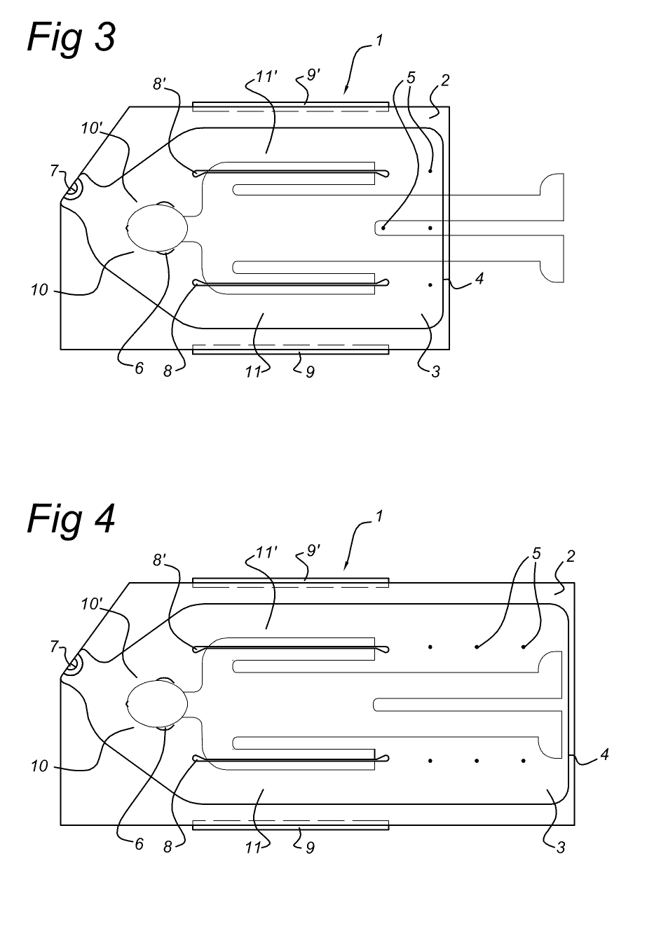

FIGS. 3 and 4 depict a projected view of a human body, lying at respectively the small blanket of FIG. 1 and the tall sized version of FIG. 2. These views clearly direct the position of the head, arms, legs and thorax of a person. The head of the person is positioned at an non-inflatable part 6 of the blanket and is flanked by the two outer channels 10, 10′.

The thermal blanket 1 includes an inflatable portion for receiving the conditioned gas under pressure and for being positioned under the body of a user. The inflatable portion is defined by the air impermeable base sheet 2 the air permeable top sheet 3. In an embodiment, one or more air permeable barrier sheets can be positioned between the top sheet 3 and base sheet 2. In use, this top sheet 3 is directed towards the body resting on the blanket. Furthermore, it is bounded by the peripheral connection 4. The inflatable portion also includes an inlet port 7, in use connected to a source of conditioned gas. The inlet port 7 is in an embodiment positioned asymmetrically with respect to the longitudinal axis 1 of the blanket as depicted at FIG. 3.

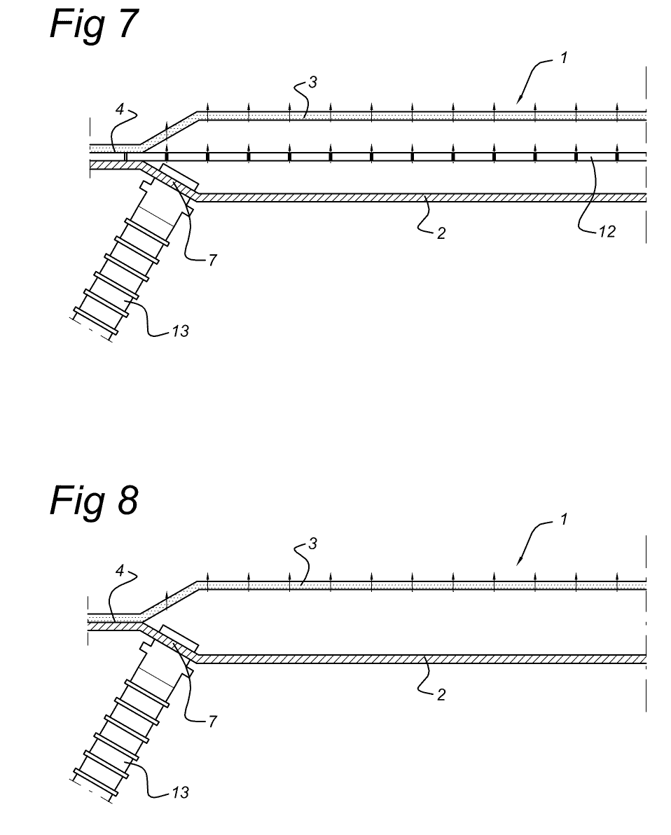

In use, the conditioned gas enters the blanket 1 via the inlet port 7 between the base sheet 2 and, if present, barrier sheet(s) 12 (see FIG. 7) or in absence of the barrier sheet(s), directly between the base sheet 2 and the top sheet 3. The barrier sheet 12 acts as a plenum, generating pressure resistance, forcing the flow of gas to distribute along the complete surface of the barrier sheet 12. In an embodiment, the air permeability of this barrier sheet about 1-400 mm/sec measured at a pressure drop of 100 Pa. This air permeability is achieved via multiple exhaust ports of any shape. Alternatively, via any porous material, for instance meltblown material or other nonwoven material.

After entering the blanket, the conditioned gas is directed to a dual inlet channel 10, 10′ configuration, creating a splitted flow to either side of the body on top of the blanket. Flow splitting is directed by a non-inflatable shaped area 6, positioning the head. In an embodiment, this non-inflatable portion 6 has a substantially triangular shape, in an embodiment with rounded edges (FIG. 2). At the non-inflatable part 6, the layers of the blanket are attached to one another using a circumferential seal, indicated in the drawings. Alternatively, the sheets of the blanket in the entire area of non-inflatable part 6 may be adhered. After passing the non-inflatable head area 6, the splitted flow from the inlet channels 10, 10′ is dispersed to two outer channels 11, 11′ and a central part, covered by the body lying on the blanket 1.

The central part in an embodiment has multiple seal connections 5 between the base sheet 2, barrier sheet(s) 12 and top sheet 3. These seal connections can be dots, strips, channels, etc., and are provided to distribute the flow of air and to control the height of the inflated blanket, in particular in combination with the non-inflatable part 6 discussed above. In fact, the current blanket can use the type of connections and pattern of connections as discussed in U.S. Ser. No. 12/342,933. This application is referred to as if fully set forth in this description.

Finally the conditioned gas leaves the inflated blanket 1 through the top layer 3 to the body via multiple exhaust ports of any shape or via any porous material as for instance meltblown material.

The flow in the outer channels 11, 11′ is directly contacting the central part by the presence of flow connections at the upstream side just at the beginning of the outer channels and at the downstream side at the end of the outer channels. How at the upstream side of the central part will warm the head, neck and upper part of the thorax; flow at the outer channels will warm the arms and thorax sides; flow at the downstream side of the central part will warm the legs and lower part of the thorax; flow at the central part will warm the thorax and arms.

In order to have multiple applications or to apply the blanket at different body sizes, the total blanket length may vary at fixed or stretched slit lengths, whilst multiplying the number of multiple connections, related to the blanket length increase.

The outer dimensions of the base and/or top sheets may be larger than the peripheral connection dimensions in order to fix the blanket to the body by fixation provisions at the peripheral longitudinal sides whilst not affecting the shape of the outer channels.

The blanket can be provided with openings 8, 8′ through with extremities like arms can pass. In an embodiment, the blanket is provided with slits. These slits can be provided near the edge of the blanket. In an embodiment shown in the drawings, the slits are elongated and are provided substantially parallel to the peripheral connection. In the embodiment shown in the drawings, the slits 8, 8′ are provided within the peripheral connection. It may be surrounded by connections 15, 15′ connecting the different sheets of the blanket together to maintain an air chamber. In an embodiment, a substantially circumferential seal 15, 15′ surrounds the slits 8, 8′ in order to prevent too much air leaving the blanket at the slits 8, 8′. In fact, the slits and the seal or peripheral connection in this embodiment provide the two outer channels 11, 11′.

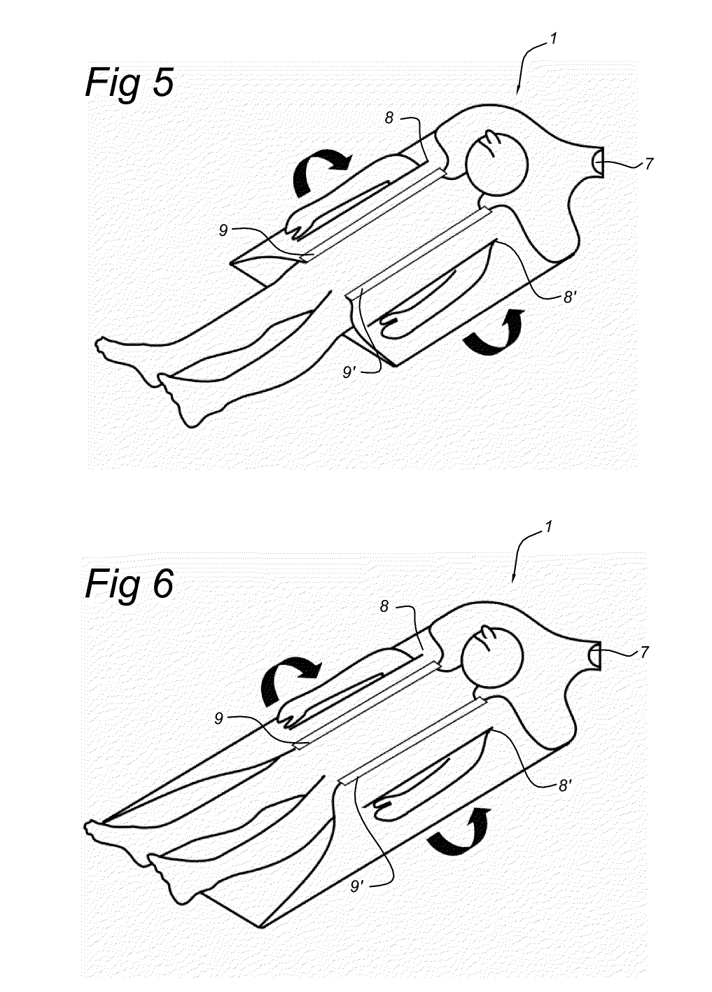

Slits 8, 8′ are provided to transfer the arms of a person through the blanket. This will prevent or flow blockage at the outer channels 11, 11′ if arms are positioned across the outer channels. Transfer of arms through the slits 8, 8′ also facilitates wrapping a patient in the blanket as depicted in FIGS. 5-6. In an embodiment, the slits can be strips of blanket which are removed, for providing a larger opening for passing for instance arms through.

In an embodiment, these slits 8, 8′ are pre-formed by cuts in the blanket. Thus, the slits 8, 8′ are selectively openable by any suitable means such as a perforated tear strip, taping, folding, sealing, snapping etc. Initially these slits are sealed closed by any means and may even comprise means to open reversibly. For instance, a removable or resealable tape may be provided over the slits 8, 8′. A different configuration may consist of permanently open slits. In an embodiment, the slits 8, 8′ have rounded ends to prevent sharp folds in the inflated blanket. The selectively openable slits or openings and the reversibly openable slits or openings also have the following use. Through one of the openings, one arm can be placed while the other opening can remain closed, allowing a body to be more completely wrapped in the blanket. The arm extending through the opening is then available for medical purposes, such as monitoring or providing an infusion.

Around the slits 8, 8′, substantially circumferential seals can be provided which attach the top sheet, base sheet, and if provided barrier sheet onto one another. The seal can comprise circumferentially provided sealing parts like dots or stripes. In an embodiment, the seal is a continuous circumferential seal or connection.

The peripheral longitudinal sides can be provided with fixing provisions 9, 9′ such as tape, velcro, push buttons etc. to maintain the sides of the inflatable thermal blanket at least partially wrapped around the body. If the arms are positioned through the slits and the blanket is fixed to the upper side of the body (FIG. 5-6), this wrapping feature creates a tight blanket configuration, resulting in a boost of temperature performance or warming effectiveness to the body. This fixation feature may also be preferable during open chest surgery were no airflow near the operating area is allowed. The fixing provision can also comprise bands extending from the longitudinal end. In an embodiment, these bands have tape, Velcro, push buttons, or other, well known provisions allowing opposite bands from the opposite longitudinal end to be fixed together. Thus, the longitudinal ends can be brought together and maintained in that position. Thus, the blanket can be more or less wrapped around a person. When the chest needs to be kept free, these bands or other fixing provisions can be provided for instance just below the head and for instance at the belly or even more downward of the body.

In fact, tests showed that wrapping a body in the blanket and closing the longitudinal sides to the body, as for instance indicated in FIGS. 5 and 6, leads to an additional temperature increase of 3 degrees Celsius.

In an embodiment, the base sheet 2 comprises a heat reflective layer. A heat reflective layer was found to increase effectiveness of the blanket.

In FIGS. 7 and 8, a cross section through part of the thermal blanket in use is shown. Via a tube or hose 13, conditioned air is provided to inlet port 7. This conditioned air fills the air chamber formed by the air permeable top sheet 3, and air impermeable base sheet 2. The air chamber is further bounded by the peripheral connection 4 which holds together the different layers forming the thermal blanket 1. In FIG. 7, an embodiment of the blanket is shown which comprises a barrier sheet 12 between said top sheet 3 and base sheet 2. In fact, it may also be possible to provide more than one barrier sheet 12. The barrier sheet 12 is provided to allow a better diffusion of conditioned gas when a body rests on the blanket 1.

In an embodiment, barrier sheet 1 is an air permeable nonwoven sheet. In an alternative embodiment, the barrier sheet 12 is a plastic sheet with openings, for instance perforations. The plastic sheet can be of any plastic material like polyethylene, polypropylene, nylon, polyester, and the like. In an embodiment, in particular in medical uses, it complies to medical requirements like being latex free and non-flammability.

In a further embodiment, the barrier sheet extends over the entire surface enclosed by the peripheral connection. The top sheet, base sheet and if present the barrier sheet, can thus be adhered to one another using heat sealing or another know sealing process or method.

In use, a person is positioned on the blanket as shown in FIG. 3 or 4. Next, the slits 8, 8′ can be opened if not already open. The persons arms are now passed through the slits 8, 8′ and the two longitudinal sides of the blanket are brought together as indicated in FIG. 5. Next, fixing provisions, like tape, can for instance be adhered to the person as indicated in FIG. 5. Alternatively, opposite fixing provisions can be fixed to one another, keeping the longitudinal sides at least partially wrapped around a person. The situations shown in FIGS. 5 and 6 show that the applied blanket forms a pipe into which warm air can be brought via inlet 7 and air permeable top sheet 3.

It will also be clear that the above description and drawings are included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

1. An inflatable thermal blanket for providing conditioned air to a human or animal body positioned on top of said thermal blanket, said thermal blanket comprising:
a base sheet of an air impermeable material, in use remote from said body;
a top sheet of an air permeable material, in use in contact with said body;
a peripheral connection joining said base sheet and top sheet to define an air chamber;
multiple connections distributed within said peripheral connection across interior surface regions of said base sheet and top sheet and connecting said base sheet and top sheet, and
an inlet port for providing the conditioned gas to said inflatable chamber.

2. The inflatable blanket of claim 1, wherein said top sheet has an air permeability of 1-400 mm/sec measured at a pressure drop of 100 Pa.

3. The inflatable blanket of claim 1, wherein the inlet port is positioned asymmetrically compared to the longitudinal axis of the blanket.

4. The inflatable blanket of claim 1, wherein said inlet port is connected to a dual inlet channel, creating a splitted flow to either side of the body.

5. The inflatable blanket of claim 4, wherein said splitted flow is directed by an non-inflatable shaped area, positioned at in use the position of the head, preferably said area is triangular shaped with rounded edges.

6. The inflatable blanket of claim 4, wherein the splitted flow is dispersed to at least one outer channel along the longitudinal end of the blanket, and a central part of the blanket.

7. The inflatable blanket of claim 6, wherein the outer channels and the central part comprise open contacts at both the upstream and downstream side of the outer channels.

8. The inflatable blanket of claim 6, wherein said central part comprises multiple seal connections between the base sheet, barrier sheet(s) and top sheet such as dots, strips, channels etc.

9. The inflatable blanket of claim 1, wherein the total blanket length varies at fixed or stretched slit lengths.

10. The inflatable blanket of claim 1 wherein the base and/or top sheets extend outward beyond said peripheral connection.

11. The inflatable blanket of claim 1, further comprising one or more barrier sheets of an air permeable material sandwiched between said base sheet and said top sheet.

12. The inflatable blanket of claim 1, wherein said one or more barrier sheets is or are attached to said top sheer and said base sheet.

13. The inflatable blanket of claim 11, wherein said barrier sheet has an air permeability of 1-400 mm/sec measured at a pressure drop of 100 Pa.

14. The inflatable thermal blanket of claim 1, further comprising openings through said top sheet and base sheet to transfer arms through said thermal blanket.

15. The inflatable thermal blanket of claim 14, wherein said openings comprise slits.

16. The inflatable thermal blanket of claim 14, wherein said openings comprise slits which are selectively openable.

17. The inflatable thermal blanket of claim 14, wherein said openings comprise slits which are selectively openable through a perforated tear strip, taping, folding, sealing, snapping, or a combination thereof.

18. The inflatable thermal blanket of claim 15, wherein said slits are initially sealed closed and are reversibly openable.

19. The blanket of claim 15, wherein said slits are permanently open.

20. The blanket of claim 15, wherein said slits have rounded ends.

21. The inflatable thermal blanket of claim 1, further comprising fixing provisions on the longitudinal sides of the inflatable thermal blanket, allowing said longitudinal end to be held towards one another.

22. The inflatable thermal blanket of claim 21, wherein said fixing provisions are adapted to adhere to the body, in particular said fixing provisions include tape.

23. The inflatable thermal blanket of claim 1, wherein said base sheet comprises a heat reflective layer.

24. A method for conditioning a person, in particular the temperature of a patient, comprising the steps of:
providing a blanket on a surface, said blanket comprising:
a base sheet of an air impermeable material, in use remote from said body;
a top sheet of an air permeable material, in use in contact with said body;
a peripheral connection joining said base sheet and top sheet to define an air chamber;
multiple connections distributed within said peripheral connection across interior surface regions of said base sheet and top sheet and connecting said base sheet and top sheet, and
an inlet port for providing the conditioned gas to said inflatable chamber, and
positioning the person on said blanket.

25. The method of claim 24, said method further comprising the step of:
folding longitudinal ends of said blanket toward one another after said person is positioned on said blanket.

26. The method of claim 25, wherein said blanket comprises openings near said longitudinal end, and said method further comprises the step of taking the arms of the person through the openings before folding said longitudinal ends.

27. The method of claim 25, wherein said blanket further comprises fixing provisions for holding said longitudinal ends near one another after folding them towards one another, said method further comprising the step of fixing said fixing provisions for fixing said longitudinal ends of said blanket positioned near one another.

28. The method of claim 26, wherein said blanket further comprises fixing provisions for holding said longitudinal ends near one another after folding them towards one another, said method further comprising the step of fixing said fixing provisions for fixing said longitudinal ends of said blanket positioned near one another.
